(19) United States
(12) Patent Application Publication
(10) Pub. No.: US 2009/0239992 A1
(43) Pub. Date: Sep. 24, 2009

(54) THERMOSETTING RESIN COMPOSITION AND UNCURED FILM COMPRISING THE SAME

(76) Inventors: Toshiaki Yamada, Niigata (JP); Hidenori Iida, Niigata (JP); Yasukazu Anbai, Niigata (JP); Shin Teraki, Niigata (JP); Masaki Yoshida, Niigata (JP); Satoko Takahashi, Niigata (JP)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 12/309,979

(22) PCT Filed: Aug. 8, 2007
(86) PCT No.: PCT/JP2007/065484

§ 371 Date: Feb. 5, 2009

(30) Foreign Application Priority Data
Aug. 8, 2006 (JP) 2006-215464

(51) Int. Cl. A61K 8/33; C08G 61/10

(52) U.S. Cl. 524/529; 525/416

(57) ABSTRACT

To provide a thermosetting resin composition having excellent film-forming properties, which can form a cured product having low elastic modulus and having low dielectric constant and low dielectric loss tangent in a high frequency range; and a thermosetting resin composition comprising (A) a vinyl compound represented by the general formula (1), and (B) a rubber and/or a thermoplastic elastomer.

### FIELD OF THE INVENTION

The present invention relates to a thermosetting resin composition having excellent film-forming properties, which can form a cured product having low elastic modulus and having low dielectric constant and low dielectric loss tangent in a high frequency range, an uncured film comprising the thermosetting resin composition, and an interlayer dielectric film for printed wiring board and an electronic part, which are obtained by using the uncured film.

### BACKGROUND ART

In the recent information oriented society highly developed, representatively, cell phones are steadily increased in frequency for achieving transmission of information in a larger capacity at a higher speed. For meeting the demand, in printed wiring boards and module substrates for use in electronic devices, such as information terminal devices, the use of a material having excellent dielectric properties such that the dielectric constant is low and the dielectric loss is low is desired.

With respect to the material having excellent dielectric properties, a curing resin composition comprising a compound of difunctional oligo phenylene ether (OPE) having ends replaced by vinyl groups has been proposed (patent document 1). However, this resin composition has a problem in that it forms a cured product which is brittle and hence unsuitable for a protective film or interlayer dielectric film for printed wiring board.

On the other hand, with respect to the material having excellent film-forming properties, a curing resin composition comprising a polyfunctional styrene compound has been proposed (patent document 2). However, this resin composition has a problem in that it forms a cured product which is not satisfactorily low in dielectric constant.
Patent document 1: Japanese Unexamined Patent Publication No. 2004-59644
Patent document 2: Japanese Unexamined Patent Publication No. 2004-83680

### DISCLOSURE OF THE INVENTION

### Problems to be Solved by the Invention

An object of the present invention is to provide a thermosetting resin composition having excellent film-forming properties such that the resin composition can form a cured product having low elastic modulus and having low dielectric constant and low dielectric loss tangent in a high frequency range, and an uncured film comprising the thermosetting resin composition. Another object of the present invention is to provide an interlayer dielectric film for printed wiring board and an electronic part which are obtained by using the uncured film. In the present specification, the film in connection with the present invention means a film which is self-supporting without being combined with a base material, such as glass cloth, glass nonwoven fabric or aramid nonwoven fabric, or without being supported by a substrate.

### Means to Solve the Problems

The present invention is directed to a thermosetting resin composition comprising: (A) a vinyl compound represented by the following general formula (1):

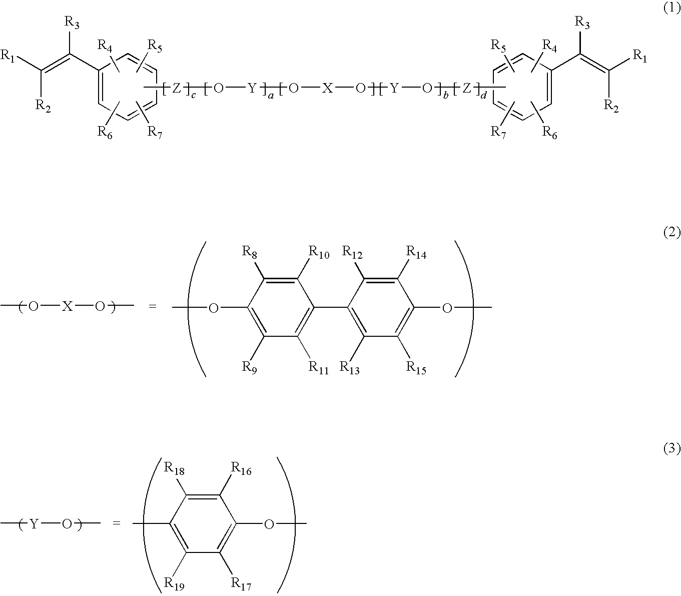

wherein:

each of R₁, R₂, R₃, R₄, R₅, R₆, and R₇ independently represents a hydrogen atom, a halogen atom, an alkyl group, a halogenated alkyl group, or a phenyl group,

—(O—X—O)— is represented by the structural formula (2) above, wherein each of R₈, R₉, R₁₀, R₁₄, and R₁₅ independently represents a halogen atom, an alkyl group having 6 or less carbon atom(s), or a phenyl group, and each of R₁₁, R₁₂, and R₁₃ independently represents a hydrogen atom, a halogen atom, an alkyl group having 6 or less carbon atom(s), or a phenyl group,

—(Y—O)— is comprised of a single structure represented by the structural formula (3) above or two or more structures represented by the structural formula (3) above, which are randomly arranged, wherein each of R₁₆ and R₁₇ independently represents a halogen atom, an alkyl group having 6 or less carbon atom(s), or a phenyl group, and each of R₁₈ and R₁₉ independently represents a hydrogen atom, a halogen atom, an alkyl group having 6 or less carbon atom(s), or a phenyl group,

Z represents an organic group having one or more carbon atom(s) and optionally contains an oxygen atom(s), a nitrogen atom(s), a sulfur atom(s), or a halogen atom(s),

each of a and b represents an integer of 0 to 300, with the proviso that at least one of a and b is not 0, and

each of c and d represents an integer of 0 or 1; and

(B) rubber and/or a thermoplastic elastomer.

### Effect of the Invention

According to the present invention, there are provided a thermosetting resin composition having excellent film-forming properties such that the resin composition can form a cured product having low elastic modulus and having low dielectric constant and low dielectric loss tangent in a high frequency range, and an uncured film comprising the thermosetting resin composition. The thermosetting resin composition or uncured film can form a cured product having low elastic modulus and having low dielectric constant and low dielectric loss tangent in a high frequency range, and therefore can be extremely advantageously used in the production of interlayer dielectric film for printed wiring board, electronic part, and others.

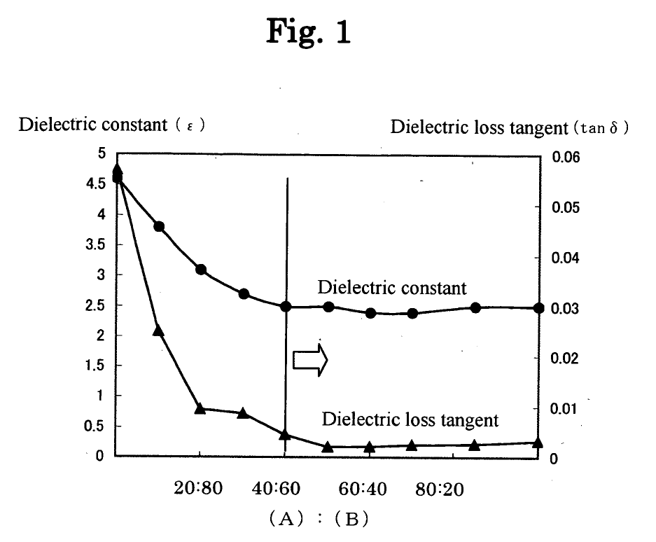

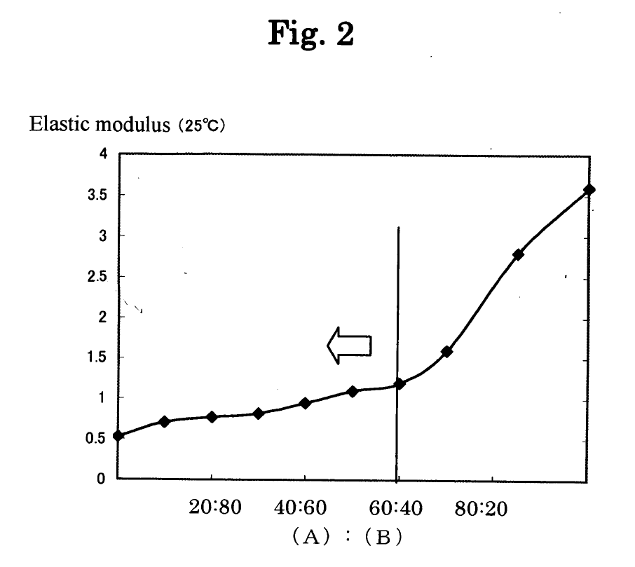

### BRIEF DESCRIPTION OF THE DRAWINGS

[FIG. 1] A graph showing the relationship between a weight ratio of component (A):component (B) and a dielectric constant or a dielectric loss tangent.

[FIG. 2] A graph showing the relationship between a weight ratio of component (A):component (B) and an elastic modulus.

### BEST MODE FOR CARRYING OUT THE INVENTION

The thermosetting resin composition of the present invention comprises, as component (A), a vinyl compound represented by the general formula (1) above. These compounds are described in Japanese Unexamined Patent Publication No. 2004-59644.

Component (A) has styrene functional groups at both ends, and therefore the thermosetting resin composition of the present invention can be easily cured by heating. From the viewpoint of achieving excellent curing properties, preferred is the vinyl compound represented by the general formula (1) wherein each of R₁ to R₇ is hydrogen.

In the structural formula (2) for —(O—X—O)— in the vinyl compound represented by the general formula (1), each of R₈, R₉, R₁₀, R₁₄, and R₁₅ is preferably an alkyl group having 3 or less carbon atom(s) (particularly, a methyl group), and each of R₁₁, R₁₂, and R₁₃ is preferably a hydrogen atom or an alkyl group having 3 or less carbon atom(s) (particularly, a methyl group). Specific examples include the following structural formula (4).

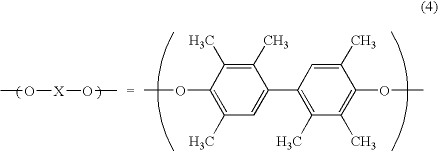

In the structural formula (3) for —(Y—O)—, each of R₁₆ and R₁₇ is preferably an alkyl group having 3 or less carbon atom(s) (particularly, a methyl group), and each of R₁₈ and R₁₉ is preferably a hydrogen atom or an alkyl group having 3 or less carbon atom(s) (particularly, a methyl group). Specific examples include the following structural formulae (5) and (6).

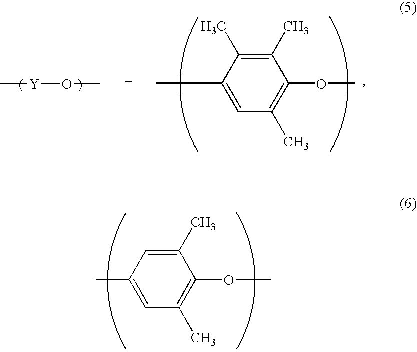

Z represents, e.g., an alkylene group having 3 or less carbon atom(s), specifically a methylene group.

Each of a and b represents an integer of 0 to 300, preferably an integer of 0 to 30, with the proviso that at least one of a and b is not 0.

For controlling the thermosetting resin composition so that the resultant cured product has an elastic modulus in an appropriate range, it is preferred that the vinyl compound of the general formula (1) has a number average molecular weight of 1,000 to 3,000. The vinyl compound of the general formula (1) preferably has at both ends functional groups each having a vinyl group and has an equivalent per functional group of 500 to 1,500 corresponding to ½ of the molecular weight mentioned above. The functional group equivalent indicates the degree of crosslink density of a cured product. When the vinyl compound has a functional group equivalent of 500 or more, a cured product having an appropriate crosslink density can be obtained to achieve satisfactory mechanical strength, making it possible to prevent, e.g., formation of a crack in the resultant film. When the vinyl compound has a functional group equivalent of 1,500 or less, the compound has excellent compatibility with component (B), so that a transparent film can be easily obtained. In addition, there can be avoided a problem in that the melt viscosity of the resin composition is increased to lower the reactivity and hence the curing temperature for the composition is inevitably increased to a high temperature disadvantageous from a practical point of view. In the present specification, the number average molecular weight is a value measured by a gel permeation chromatography (GPC) method using a calibration curve obtained from standard polystyrenes.

The vinyl compound of the general formula (1) can be prepared by the method described in Japanese Unexamined Patent Publication No. 2004-59644. For example, there can be used a reaction product obtained by reacting a polycondensation product of 2,2′,3,3′,5,5′-hexamethylbiphenyl-4,4′-diol and 2,6-dimethylphenol with chloromethylstyrene.

The vinyl compounds of the general formula (1) above can be used individually or in combination.

The thermosetting resin composition of the present invention comprises, as component (B), a rubber and/or a thermoplastic elastomer. Examples of rubbers include a styrene-butadiene rubber, a butyl rubber, a butadiene rubber, and an acrylic rubber, and preferred is a styrene-butadiene rubber.

Examples of thermoplastic elastomers include styrene thermoplastic elastomers, such as a styrene-butadiene-styrene block copolymer (SBS) and a styrene-isoprene-styrene block copolymer (SIS), olefin thermoplastic elastomers, and polyester thermoplastic elastomers.

From the viewpoint of obtaining a cured product having low elastic modulus, preferred is a thermoplastic elastomer. Especially when used in, e.g. interlayer dielectric film, from the viewpoint of obtaining a cured product having excellent burying properties and low dielectric constant, preferred is a styrene thermoplastic elastomer. A styrene thermoplastic elastomer having a weight average molecular weight of 20,000 to 250,000 can be used, and, from the viewpoint of achieving a thermosetting resin composition having excellent film-forming properties, the styrene thermoplastic elastomer preferably has a weight average molecular weight of 30,000 to 150,000, more preferably 50,000 to 130,000. In the present specification, the weight average molecular weight is a value measured by a gel permeation chromatography (GPC) method using a calibration curve obtained from standard polystyrenes.

From the viewpoint of achieving excellent compatibility with component (A) and obtaining a film having excellent transparency, the styrene thermoplastic elastomer preferably has a styrene content of 25 to 60% by weight, more preferably 30 to 50% by weight.

With respect to the styrene thermoplastic elastomer, a diblock-type or triblock-type elastomer can be used. From the viewpoint of achieving a thermosetting resin composition having excellent film-forming properties and obtaining a cured product having excellent dielectric properties and low elastic modulus, preferred is a triblock-type elastomer. Examples of triblock-type elastomers include a styrene-butadiene-styrene block copolymer (SBS), a styrene-isoprene-styrene block copolymer (SIS), a styrene-(ethylene•butylene)-styrene block copolymer (SEBS), a styrene-(ethylene•propylene)-styrene block copolymer (SEPS), a styrene-(butadiene•butylene)-styrene block copolymer (SBBS), and a styrene-(ethylene•ethylene-propylene)-styrene block copolymer (SEEPS). Of these, preferred is a styrene-butadiene-styrene block copolymer (SBS) or a styrene-(ethylene•butylene)-styrene block copolymer (SEBS), and, from the viewpoint of easily controlling the cured product so that it has a glass transition temperature in an appropriate range and obtaining a cured product having excellent bonding properties with a copper foil and excellent shear strength even at high temperatures, especially preferred is a styrene-butadiene-styrene block copolymer (SBS).

When a styrene-butadiene-styrene block copolymer (SBS) is used, from the viewpoint of achieving excellent compatibility between component (A) and component (B) and excellent film-forming properties, and obtaining a cured product having excellent strength balance, the copolymer preferably has a weight average molecular weight of 30,000 to 150,000 and a styrene content of 25 to 60% by weight, especially preferably a weight average molecular weight of 60,000 to 120,000 and a styrene content of 30 to 50% by weight. When a styrene-(ethylene•butylene)-styrene block copolymer (SEBS) is used, from the viewpoint of achieving excellent compatibility between component (A) and component (B) and excellent film-forming properties, and obtaining a cured product having excellent strength balance, the copolymer preferably has a weight average molecular weight of 30,000 to 150,000 and a styrene content of 25 to 60% by weight, especially preferably a weight average molecular weight of 70,000 to 120,000 and a styrene content of 30 to 50% by weight.

The rubber and/or thermoplastic elastomer can be used individually or in combination.

It is preferred that a weight ratio of the component (A):component (B) is 10:90 to 90:10. For obtaining a cured product having good balance between low dielectric constant, low dielectric loss tangent, and low elastic modulus, it is more preferred that a weight ratio of the component (A):component (B) is 40:60 to 60:40.

An additive, such as inorganic filler, a tackifier, an anti-foaming agent, a flow modifier, a film-forming auxiliary, or a dispersing agent, can be added to the thermosetting resin composition of the present invention in such an amount that the desired effects of the present invention are not sacrificed. An aromatic vinyl compound other than component (A) can be added to the thermosetting resin composition, and examples of such aromatic vinyl compounds include α-methylstyrene, vinyltoluene, chlorostyrene, and divinylbenzene. Examples of alicyclic vinyl compounds include cyclohexene, 4-vinylcyclohexene, and 1,5-cyclooctadiene. Unsaturated fatty acids having a vinyl group and derivatives thereof include those which are monofunctional and those which are polyfunctional, and examples include methyl (meth)acrylate, ethyl (meth)acrylate, octadecyl (meth)acrylate, cyclohexyl (meth)acrylate, isobornyl (meth)acrylate, phenyl (meth)acrylate, chlorophenyl (meth)acrylate, benzyl (meth)acrylate, 2-hydroxyethyl (meth)acrylate, polyethylene glycol (meth)acrylate, N,N-dimethyl(meth)acrylamide, (meth)acrylic acid, ethylene glycol di(meth)acrylate, diethylene glycol di(meth)acrylate, tetraethylene glycol di(meth)acrylate, propylene glycol di(meth)acrylate, tripropylene glycol di(meth)acrylate, 1,6-hexanediol di(meth)acrylate, trimethylolpropane tri(meth)acrylate, pentaerythritol tri(meth)acrylate, and pentaerythritol tetra(meth)acrylate. Examples of vinyl ether compounds include ethyl vinyl ether, n-propyl vinyl ether, isobutyl vinyl ether, n-amyl vinyl ether, cyclohexyl vinyl ether, ethylene glycol monovinyl ether, diethylene glycol monovinyl ether, diethylene glycol divinyl ether, triethylene glycol monovinyl ether, cyclohexanedimethanol vinyl ether, and phenyl vinyl ether.

The thermosetting resin composition of the present invention can be cured merely by heating without using a curing catalyst, but a curing catalyst can be used in the composition. With respect to the curing catalyst which generates cations or radical active species capable of initiating the polymerization of styrene group by heat or light, examples of cationic polymerization initiators include diallyliodonium salts, triallylsulfonium salts, and aliphatic sulfonium salts, each having BF₄, PF₆, AsF₆, or SbF₆ as counter anion, and specific examples include SP-70, 172, CP-66, manufactured by Asahi Denka Kogyo K.K.; CI-2855, 2823, manufactured by NIPPON SODA CO., LTD.; and SI-100L and SI-150L, manufactured by SANSHIN CHEMICAL INDUSTRY CO. LTD. Examples of radical polymerization initiators include benzoin compounds, such as benzoin and benzoin methyl; acetophenone compounds, such as acetophenone and 2,2-dimethoxy-2-phenylacetophenone; thioxanthone compounds, such as thioxanthone and 2,4-diethylthioxanthone; bisazide compounds, such as 4,4′-diazidochalcone, 2,6-bis(4′-azidobenzal)cyclohexanone, and 4,4′-diazidobenzophenone; azo compounds, such as azobisisobutyronitrile, 2,2-azobispropane, m,m′-azoxystyrene, and hydrazone; and organic peroxides, such as 2,5-dimethyl-2,5-di(t-butylperoxy)hexane, 2,5-dimethyl-2,5-di(t-butylperoxy)hexyne-3, and dicumyl peroxide.

A varnish can be prepared by dissolving or dispersing the thermosetting resin composition of the present invention in an organic solvent. An uncured film can be formed by applying the resultant varnish to a desired substrate and drying the varnish.

Examples of organic solvents include aromatic solvents, such as toluene and xylene, and ketone solvents, such as methyl ethyl ketone and methyl isobutyl ketone. Considering influence of the residual solvent on the dielectric properties, preferred is an aromatic solvent, such as toluene or xylene. The organic solvents can be used individually or in combination. With respect to the amount of the organic solvent used, there is no particular limitation, and the resin composition can be diluted with the solvent in such an amount that the resultant varnish has a viscosity appropriate to the application. When using a general application method, it is preferred that the viscosity of the varnish is adjusted to a range of 100 to 600 mpa·s. In this case, in component (A) and component (B), generally, the resin solids content is 20 to 50% by weight. The viscosity is a value measured at 25° C. at a number of revolutions of 60 rpm using an E-type viscometer.

In the present invention, it is important to select component (B) compatible with component (A). Specifically, it is considered that components (A) and (B) having excellent compatibility are unlikely to be separated from each other even when the resin concentration of the composition is increased due to volatilization of the solvent in the drying step, thus forming a uniform film. Attention was drawn to the fact that acetone is a good solvent for a general oligo phenylene ether and a poor solvent for a general polymer elastomer, and studies were made. As a result, it has been found that a solubility stability test for component (A) and component (B) in a mixed solvent comprising 85 parts by weight of toluene and 15 parts by weight of acetone can be advantageously used as a yardstick for the compatibility. It is considered that this test facilitates the selection of component (B) having a molecular structure compatible with component (A) in the presence of acetone. Specifically, in case where component (A) and component (B) are combined so that a transparent solution is obtained when a varnish (having a solids content of 50% by weight) is prepared from component (A) and component (B) in a 50/50 weight ratio using a mixed solvent comprising 85 parts by weight of toluene and 15 parts by weight of acetone, excellent compatibility is expected. When component (B) is a triblock-type styrene thermoplastic elastomer, it is presumed that the solubility of the elastomer in a solvent is possibly affected by not only the type or molecular weight of the elastomer but also the symmetry of hard segments (styrene blocks) at both ends having disposed therebetween a soft segment comprised mainly of a conjugated diene. The symmetry of the hard segments (styrene blocks) of component (B) at both ends is a factor that possibly affects the properties of the uncured film comprising the thermosetting resin composition of the present invention, and it is presumed that, when the styrene blocks at both ends have different molecular weights and hence are asymmetric, the uncured film is improved in, e.g., bonding properties.

With respect to the substrate, there is no particular limitation, and examples include a metallic foil comprised of copper, aluminum, or an ITO film, and an organic film comprised of a polyester resin or a polyethylene resin. The substrate may be treated by release agent, e.g., by a silicone compound. The uncured film comprising the thermosetting resin composition of the present invention is self-supporting and hence can be used in the form of a film itself removed from the substrate.

With respect to the method for applying a varnish, there is no particular limitation, but, from the viewpoint of forming a film having a reduced thickness or controlling the film thickness, a microgravure method or a slot die method is preferred. By a microgravure method, for example, an uncured film having a final thickness of 90 μm or less, e.g., 2 to 90 μm can be obtained.

Conditions for drying the varnish can be appropriately selected depending on the type or amount of the organic solvent used in the varnish or the thickness of the varnish applied, and the varnish can be dried, e.g., at 80 to 120° C. for about 1 to 30 minutes.

Thus an uncured film in an uncured state can be obtained. The uncured film comprising the thermosetting resin composition of the present invention has excellent storage stability.

The uncured film can be further cured. Conditions for curing the uncured film can be appropriately selected, and the uncured film can be cured, e.g., at 150 to 250° C. for about 10 to 120 minutes.

The cured product has low dielectric constant and low dielectric loss tangent in a high frequency range, and, for example, the cured product can achieve a dielectric constant of 4 or less and a dielectric loss tangent of 0.025 or less under conditions such that the temperature is 25° C. and the frequency is 5 GHz. Further, for example, by appropriately controlling the ratio of the amounts of components (A) and (B), the cured product can achieve a dielectric constant of 2.6 or less and a dielectric loss tangent of 0.005 or less.

The cured product has low elastic modulus and contributes to stress relaxation, and can be easily handled during the processing. For example, the cured product can achieve an elastic modulus of 3.5 GPa or less at a temperature of 25° C. as measured by dynamic viscoelastic analysis {at a frequency of 10 Hz (tensile mode)}. Further, for example, by appropriately controlling the ratio of the amounts of components (A) and (B), the cured product can achieve an elastic modulus of 1.5 GPa or less. Particularly, for forming a film having a thickness as small as 30 μm or less without causing a crack in the film, the cured product preferably has an elastic modulus of 0.4 to 2.7 GPa. When the elastic modulus is 0.4 GPa or more, satisfactory hot shear strength can be obtained. Further, when the elastic modulus is 2.7 or less, there can be avoided a disadvantage in that the resultant film is so brittle that a crack is likely to be formed in the film. In this case, the cured product preferably has a glass transition temperature of 150° C. or higher. The cured product more preferably has an elastic modulus in the range of 0.7 to 2.3 GPa.

Especially when a styrene-butadiene-styrene block copolymer (SBS) is used as component (B) and a weight ratio of the component (A):component (B) is in the range of from 40:60 to 60:40, there can be obtained an uncured film having such excellent form that a crack is unlikely to be formed in the film even when the film is bent. When the uncured film is formed on a substrate, high peel strength is achieved, and, for example, a film with copper foil having excellent bonding properties can be obtained. Further, when film a silicon chip is bonded by the uncured, it exhibits excellent shear strength. The uncured film can be easily controlled so that it has a glass transition temperature in an appropriate range, e.g., 140 to 180° C.

When a weight ratio of the component (A):component (B) is 60/40 or less, the cured product can be easily controlled so that it has an elastic modulus of 1.5 GPa or less, e.g., 0.9 to 1.5 GPa. When a weight ratio of the component (A):component (B) is 40/60 or more, the cured product can be easily controlled so that it has a dielectric constant of 2.6 or less and a dielectric loss tangent of 0.005 or less.

The uncured film comprising the thermosetting resin composition of the present invention can be used in the preparation of printed wiring board or module substrate (e.g., preparation of interlayer dielectric film for printed wiring board) or bonding in the preparation process for electronic part (e.g., bonding different materials). Using a metallic foil as a substrate, a film with metallic foil (e.g., a film with copper foil) can be formed from the uncured film.

For example, when the uncured film is used, e.g., in bonding different materials or in interlayer dielectric film, the uncured film is placed on a desired substrate and then can be cured by vacuum press. Conditions for the vacuum press can be appropriately selected, for example, such that the temperature is 170 to 210° C. and the actual pressure is 5 to 15 kgf/cm². The lowest melt viscosity of the uncured film can be relatively high (e.g., 1,000 to 10,000 Pa·s under conditions at a holding temperature of 170 to 210° C.), and therefore not only can the resin be prevented from flowing during the vacuum press to render substantially constant the thickness of the film before and after being cured, but also the thickness of the film cured can be uniform.

The uncured film comprising the thermosetting resin composition of the present invention forms a cured product having low dielectric constant and low dielectric loss tangent in a high frequency range, and therefore the uncured film is advantageously used particularly in the preparation of an electronic part used in a high frequency range. Further, the uncured film formed from the thermosetting resin composition of the present invention has excellent burying properties, and is excellent in processability, e.g., perforation using laser. In addition, the resultant cured film is free of uneven thickness, and hence advantageously used particularly in the production of, e.g., interlayer dielectric film for printed wiring board.

### EXAMPLES

Hereinbelow, the present invention will be described in more detail with reference to the following Examples, which should not be construed as limiting the scope of the present invention. The indications for amount are part(s) by weight unless otherwise specified.

A varnish of resin composition having the formulation shown in Table 1 (solids content: about 30% by weight) was prepared using methyl ethyl ketone as a solvent. The resultant varnish was applied to a substrate (PET) by means of a microgravure coater so that the thickness became 30 μm, and then dried under conditions at 80 to 120° C. for 10 minutes to obtain an uncured film. The thus obtained uncured film was cured under conditions at 200° C. for 60 minutes to form a sample.

The cured film was cut into a 40 mm×100 mm size, and shaped into a form of cylinder having a diameter of about 2 mm, and a dielectric constant (ε) and a dielectric loss tangent (tan δ) were measured using a cavity resonator at a temperature of 25° C. at a frequency of 5 GHz. The results are shown in Table 1 and FIG. 1.

A glass transition temperature (Tg) and an elastic modulus were measured by dynamic viscoelastic analysis (DMA) at a frequency of 10 Hz (tensile mode). The results are shown in Table 1 and FIG. 2.

TABLE 1

| | Comparative | | | | | | | | | Comparative |
|---|---|---|---|---|---|---|---|---|---|---|
| | Example 1 | Example 1 | Example 2 | Example 3 | Example 4 | Example 5 | Example 6 | Example 7 | Example 8 | Example 2 |
| (A) Vinyl compound*1 | 100 | 85 | 70 | 60 | 50 | 40 | 30 | 20 | 10 | 0 |
| (B) Styrene-butadiene- | 0 | 15 | 30 | 40 | 50 | 60 | 70 | 80 | 90 | 100 |
| styrene block | | | | | | | | | | |
| copolymer*2 | | | | | | | | | | |
| Tg (° C.) | 235 | 187 | 169 | 170 | 172 | 152 | 138 | 113 | 77 | 65 |
| Elastic modulus Gpa | 3.6 | 2.8 | 1.6 | 1.2 | 1.1 | 0.95 | 0.82 | 0.77 | 0.71 | 0.53 |
| (25° C.) | | | | | | | | | | |
| ε | 2.5 | 2.5 | 2.4 | 2.4 | 2.5 | 2.5 | 2.7 | 3.1 | 3.8 | 4.6 |
| tan δ | 0.0032 | 0.0026 | 0.0024 | 0.0021 | 0.002 | 0.0045 | 0.0087 | 0.0095 | 0.023 | 0.057 |
| Curing temperature | 200 | 200 | 200 | 200 | 200 | 200 | 200 | 200 | 200 | — |
| (° C.) | | | | | | | | | | |

*1Trade name: OPE-2st, manufactured by MITSUBISHI GAS CHEMICAL COMPANY, INC. {General formula (1) wherein each of R₁ to R₇ is hydrogen, —(O—X—O) - is the structural formula (4), —(Y—O) - is the structural formula (6), z is a methylene group, and each of c and d is 1; number average molecular weight: 2,200}
*2Trade name: TR2003, manufactured by JSR Corporation

The cured film used in Example 4 was stored for 90 days under conditions such that the temperature was 25° C. and the relative humidity was 60%, and then the film was cured in the same manner as in the above method, and a dielectric constant (ε) and a dielectric loss tangent (tan δ) were measured. As a result, it was found that they were respectively 2.5 and 0.002, which had not changed from the values of the film before stored.

Using the varnish used in Example 4, an uncured film having a thickness of 56 μm was obtained in the same manner as in the above method. Then, this uncured film was laminated on an FR-4 substrate with copper foil (size: 180 mm×180 mm), followed by high-temperature vacuum press. Conditions for the press were such that the temperature elevation rate was 8° C./minute, the temperature was 180° C., the actual pressure was 10 kgf/cm², and the holding time was 90 minutes. With respect to each of the FR-4 substrate before being treated and the FR-4 substrate having a cured product after the high-temperature vacuum press, a thickness of the cured product was measured for the same portions (16 portions ). As a result, it was found that any portions had a thickness of 54 to 55 μm.

The above cured product was processed using laser to form a 50-μm via. A via was formed without causing a crack or the like in the cured product. The above film was individually laminated on an FR-4 substrate with pattern having a line/space of 500/500 μm or 30/30 μm in the same manner as in the above method, followed by high-temperature vacuum press. As a result, it was found that the film had excellent burying properties.

From the results of Examples 1 to 8 shown in Table 1, it is apparent that the thermosetting resin composition of the present invention forms a cured product having low dielectric constant and low dielectric loss tangent as well as low elastic modulus. Particularly, from the results of Examples 3 to 5, it is apparent that, when the ratio of components (A) and (B) is in the range of from 40:60 to 60:40, the balance between the low dielectric constant, low dielectric loss tangent, and low elastic modulus is especially excellent. Further, it is found that the film obtained by using the thermosetting resin composition of the present invention has such excellent storage stability that the film after being stored for 90 days can form a cured product having low dielectric constant and low dielectric loss tangent. In addition, the film obtained by using the thermosetting resin composition of the present invention has excellent burying properties, and is excellent in processability, e.g., perforation using laser. Further, it is found that the thickness of the film before and after being cured is substantially constant and the thickness of the film cured is excellent in thickness uniformity.

With respect to each of the resin compositions in Examples 2 to 6, the following values were measured. The results are shown in Table 2.

### (1) Compatibility

A varnish of resin composition having the formulation for Examples 2 to 6 shown in Table 1 was prepared (solids content: about 30% by weight) using toluene as a solvent. The resultant varnish was allowed to stand at room temperature, and visually checked whether phase separation or turbidity occurred in the varnish or not. With respect to the varnish in which phase separation or turbidity did not occur, the varnish was applied to a polyethylene terephthalate film (PET film) using a doctor blade (clearance: 100 to 150 μm), and dried at 100° C. for 5 minutes to form an uncured film having a thickness of 25 μm, and the film was visually checked whether the film had haze or not.

◯: Film has no haze.

Δ: Varnish has no turbidity. Film has haze.

X: Phase separation or turbidity occurs in varnish.

Film is not formed from the varnish.

### (2) Film Crack

An uncured film was formed in the same manner as in the compatibility test above, and bent at 90 degrees, together with the PET film, and visually checked whether a crack was caused in the film or not.

(3) Peel Strength

An uncured film was formed in the same manner as in the compatibility test above, and the PET film was removed, and the uncured film was disposed between electrolytic copper foils (thickness: 18 μm; roughened surface roughness Rz: 1.8 μm; gloss surface roughness Rz: 0.25 μm) so that the roughened surfaces or gloss surfaces of the copper foils faced each other, followed by heating and pressing under vacuum (200° C.; 60 minutes; 1 MPa; degree of vacuum: 1.0 KPa). The resultant sample was cut into a width of 10 mm and a peel strength was measured in accordance with JIS C5016.

(4) Shear Strength

An uncured film was formed in the same manner as in the compatibility test above, and heated to 150° C. and placed on a silicon chip 2 mm square to provisionally bond them together. Then, the PET film was removed, and the uncured film was placed on an FR-4 substrate heated to 180° C. The film placed on the substrate was cured by heating at 200° C. for 60 minutes, and then cooled to room temperature to obtain a sample. With respect to the sample obtained, using a bond tester (model: Universal Bond Tester Series 4000; manufactured by DAGE ARCTEK CO., LTD.), a shear strength was measured at a shear rate of 0.1 mm/second at room temperature or 180° C.

In Comparative Examples 1 and 2, a shear strength was measured at room temperature in substantially the same manner as in the Examples except that samples were individually prepared as follows.

In Comparative Example 1, a varnish (solids content: about 30% by weight) was prepared using toluene as a solvent, and then directly applied to an FR-4 substrate and dried to form a film having a thickness of about 25 μm. Then, the film was heated to 150° C., and the film in a molten state was placed on a silicon chip 2 mm square and then cured by heating at 200° C. for 60 minutes, and then cooled to room temperature to obtain a sample.

In Comparative Example 2, a varnish (solids content: about 30% by weight) was prepared using toluene as a solvent, and then directly applied to an FR-4 substrate and dried to form a film having a thickness of about 25 μm. Then, the film was heated to 150° C., and the film in a molten state was placed on a silicon chip 2 mm square, and then cooled to obtain a sample.

TABLE 2

| | | Comparative | | | | | | Comparative |
|---|---|---|---|---|---|---|---|---|
| | | Example 1 | Example 2 | Example 3 | Example 4 | Example 5 | Example 6 | Example 2 |
| (A):(B) | | 100:0 | 70:30 | 60:40 | 50:50 | 40:60 | 30:70 | 0:100 |
| Compatibility | | — | ◯ | ◯ | ◯ | ◯ | ◯ | — |
| Film crack | | — | Caused | Not caused | Not caused | Not caused | Caused | — |
| Peel | Roughened surface | — | 8.9 | 12.3 | 12.7 | 14.2 | 16.4 | — |
| strength | [N/cm] | | | | | | | |
| | Gloss surface | — | 4.3 | 6.5 | 7.3 | 8.0 | 8.7 | — |
| | [N/cm] | | | | | | | |
| Shear | Room temperature | 1.2 | 65.2 | 73.2 | 81.4 | 84.7 | 86.2 | 14.0 |
| strength | [N/2 mm□] | | | | | | | |
| | 180° C. [N/2 mm□] | — | 50.7 | 71.5 | 80.1 | 78.2 | 73.3 | — |

From a comparison between the results of Comparative Examples 1 and 2 and the results of Examples 2 to 6 shown in Table 2, it is apparent that there is synergy between component (A) and component (B) and the combination of components (A) and (B) drastically improves the shear strength, as compared to the use of the individual single component.

From the results of Examples 3 to 5 shown in Table 2, it is apparent that, when the (A):(B) ratio is 40:60 to 60:40, a crack is not formed in the film even being bent and the cured product has excellent balance between the peel strength and shear strength. Particularly, it is found that, when the (A):(B) ratio is in this range, the cured product exhibits excellent shear strength even at a high temperature.

A varnish of resin composition having the formulation shown in Table 3 was prepared (solids content: about 30% by weight) using toluene as a solvent. Using the resultant varnish, a sample was prepared in the same manner as in Examples 1 to 8, and a glass transition temperature (Tg) and an elastic modulus were measured by dynamic viscoelastic analysis (DMA) at a frequency of 10 Hz (tensile mode). The results are shown in Table 3.

Further, compatibility, film crack, a peel strength, and a shear strength were measured in the same manner as in Examples 2 to 6. The results are shown in Table 3.

TABLE 3

| | | Exam- | Exam- | Exam- |
|---|---|---|---|---|
| | | ple 9 | ple 10 | ple 11 |
| Vinyl compound 1 | | 50 | — | — |
| Vinyl compound 2 | | — | 50 | 50 |
| SBS (Mw: 100,000; styrene | | — | — | 50 |
| content: 43 wt %) | | | | |
| SEBS (Mw: 100,000; styrene | | 50 | 50 | — |
| content: 30 wt %) | | | | |
| Tg (° C.) | | 207 | 208 | 175 |
| Elastic modulus Gpa (25° C.) | | 0.58 | 0.6 | 1.3 |
| ε | | 2.4 | 2.4 | 2.4 |
| tan δ | | 0.0019 | 0.0017 | 0.0019 |
| Compatibility | | ∘ | ∘ | ∘ |
| Film crack | | Not | Not | Not |
| | | caused | caused | caused |
| Peel | Roughened surface | 10.2 | 10.6 | 13.5 |
| strength | [N/cm] | | | |
| | Gloss surface | 5.9 | 5.7 | 7.4 |
| | [N/cm] | | | |
| Shear | Room temperature | 66.2 | 71.1 | 85.1 |
| strength | [N/2 mm□] | | | |
| | 180° C. [N/2 mm□] | 11.0 | 12.5 | 83.7 |

Vinyl compound 1:
Trade name: OPE-2st 2200, manufactured by MITSUBISHI GAS CHEMICAL COMPANY, INC.
{General formula (1) wherein each of R₁ to R₇ is hydrogen, —(O—X—O)— is the structural formula (4), —(Y—O)— is the structural formula (6), z is a methylene group, and each of c and d is 1; number average molecular weight: 2,200; equivalent per functional group: 1,100 g/eq.}
Vinyl compound 2:
Trade name: OPE-2st 1200, manufactured by MITSUBISHI GAS CHEMICAL COMPANY, INC.
{General formula (1) wherein each of R₁ to R₇ is hydrogen, —(O—X—O)— is the structural formula (4), —(Y—O)— is the structural formula (6), z is a methylene group, and each of c and d is 1; number average molecular weight: 1,200; equivalent per functional group: 600 g/eq.}

From the results of Examples 9 to 11 shown in Table 3, it is apparent that the thermosetting resin composition of the present invention forms a cured product having low dielectric constant and low dielectric loss tangent as well as low elastic modulus. Particularly, from the results of Example 11 using SBS, it is apparent that the cured product exhibits excellent shear strength even at a high temperature.

With respect to the formulations shown in Table 4, compatibility and film crack were measured in the same manner as in Examples 2 to 6. Further, in Examples 12 to 18, a varnish (solids content: 50% by weight) was prepared from component (A) and component (B) in a 50/50 weight ratio using a mixed solvent comprising 85 parts by weight of toluene and 15 parts by weight of acetone, and the appearance of the varnish was observed. With respect to the solubility stability, a varnish in the state of transparent liquid was rated symbol “◯”, and a varnish in which turbidity or phase separation occurred was rated symbol “Δ”.

TABLE 4

| | Example | Example | Example | Example | Example | Example | Example |
|---|---|---|---|---|---|---|---|
| | 12 | 13 | 14 | 15 | 16 | 17 | 18 |
| Vinyl compound 1 | 50 | 50 | 50 | 50 | 50 | 50 | 50 |
| SEBS | 50 | | | | | | |
| (Mw70000; styrene content: 20 wt %) | | | | | | | |
| SBS | | 50 | | | | | |
| (Mw130000; styrene content: 24 wt %) | | | | | | | |
| SEBS | | | 50 | | | | |
| (Mw100000; styrene content: 30 wt %) | | | | | | | |
| SEBS | | | | 50 | | | |
| (Mw70000; styrene content: 30 wt %) | | | | | | | |
| SEBS | | | | | 50 | | |
| (Mw70000; styrene content: 42 wt %) | | | | | | | |
| SBS | | | | | | 50 | |
| (Mw100000; styrene content: 43 wt %) | | | | | | | |
| SEBS | | | | | | | 50 |
| (Mw200000; styrene content: 67 wt %) | | | | | | | |
| Compatibility | Δ | Δ | ◯ | ◯ | ◯ | ◯ | Δ |
| Solubility stability | ◯ | Δ | ◯ | ◯ | ◯ | ◯ | Δ |
| Film crack | Not | Not | Not | caused | caused | Not | Not |
| | caused | caused | caused | | | caused | caused |

Vinyl compound 1: Trade name: OPE-2st 2200, manufactured by MITSUBISHI GAS CHEMICAL COMPANY, INC. {General formula (1) wherein each of R₁ to R₇ is hydrogen, —(O—X—O) - is the structural formula (4), —(Y—O) - is the structural formula (6), z is a methylene group, and each of c and d is 1; number average molecular weight: 2,200; equivalent per functional group: 1,100 g/eq.}

From the results of Examples 14 to 17 shown in Table 4, it is apparent that, when the styrene content is in the range of from 25 to 60% by weight, a transparent film free of haze can be obtained.

1. A thermosetting resin composition comprising:
(A) a vinyl compound represented by the following general formula (1): 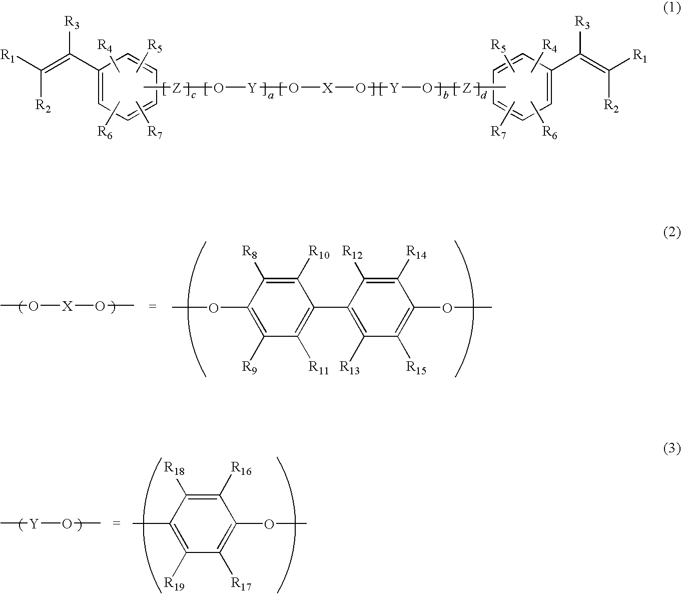
wherein:
each of R₁, R₂, R₃, R₄, R₅, R₆, and R₇ independently represents a hydrogen atom, a halogen atom, an alkyl group, a halogenated alkyl group, or a phenyl group,
—(O—X—O)— is represented by the structural formula (2), wherein each of R₈, R₉, R₁₀, R₁₄, and R₁₅ independently represents a halogen atom, an alkyl group having 6 or less carbon atom(s), or a phenyl group, and each of R₁₁, R₁₂, and R₁₃ independently represents a hydrogen atom, a halogen atom, an alkyl group having 6 or less carbon atom(s), or a phenyl group,
—(Y—O)— is comprised of a single structure represented by the structural formula (3), or two or more structures represented by the structural formula (3) which are randomly arranged, wherein each of R₁₆ and R₁₇ independently represents a halogen atom, an alkyl group having 6 or less carbon atom(s), or
a phenyl group, and each of R₁₈ and R₁₉ independently represents a hydrogen atom, a halogen atom, an alkyl group having 6 or less carbon atom(s), or a phenyl group,
Z represents an organic group having one or more carbon atom(s) and optionally contains an oxygen atom(s), a nitrogen atom(s), a sulfur atom(s), or a halogen atom(s),
each of a and b represents an integer of 0 to 300, with the proviso that at least one of a and b is not 0, and
each of c and d represents an integer of 0 or 1; and
(B) at least one of a rubber and a thermoplastic elastomer.

2. The thermosetting resin composition according to claim 1, wherein the component (A) is a vinyl compound represented by the general formula (1) wherein the —(O—X—O)— is represented by the structural formula (4) and the —(Y—O)— has a structure represented by the structural formula (5) or structural formula (6), or structures represented by the structural formula (5) and structural formula (6) which are randomly arranged.

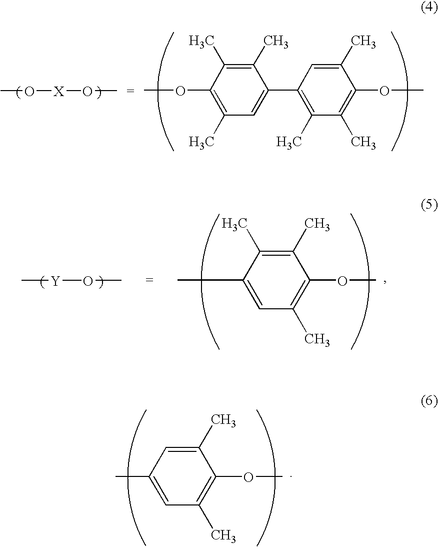

3. The thermosetting resin composition according to claim 2, wherein the —(Y—O)— has a structure represented by the structural formula (6).

4. The thermosetting resin composition according to claim 1, wherein the component (A) is a vinyl compound represented by the general formula (1), having at both ends functional groups each having a vinyl group, and having an equivalent per functional group of 500 to 1,500 g/eg.

5. The thermosetting resin composition according to claim 1, wherein a weight ratio of the component (A):component (B) is 40:60 to 60:40.

6. The thermosetting resin composition according to claim 5, wherein the component (B) is a styrene thermoplastic elastomer.

7. The thermosetting resin composition according to claim 6, wherein the component (B) is a styrene thermoplastic elastomer having a weight average molecular weight of 20,000 to 250,000.

8. The thermosetting resin composition according to claim 7, wherein the component (B) is a styrene thermoplastic elastomer having a styrene content of 25 to 60% by weight.

9. The thermosetting resin composition according to claim 8, wherein the component (B) is a triblock-type styrene thermoplastic elastomer selected from the group consisting of a styrene-butadiene-styrene block copolymer (SBS), a styrene-isoprene-styrene block copolymer (SIS), a styrene-(ethylene•butylene)-styrene block copolymer (SEBS), a styrene-(ethylene•propylene)-styrene block copolymer (SEPS), a styrene-(butadiene•butylene)-styrene block copolymer (SBBS), and a styrene-(ethylene•ethylene-propylene)-styrene block copolymer (SEEPS).

10. The thermosetting resin composition according to claim 9, wherein the component (B) is selected from the group consisting of a styrene-butadiene-styrene block copolymer (SBS) and a styrene-(ethylene•butylene)-styrene block copolymer (SEBS).

11. An uncured film comprising the thermosetting resin composition according to claim 1.

12. The uncured film according to claim 11 obtained by applying a varnish comprising the thermosetting resin composition to a substrate followed by drying.

13. The uncured film according to claim 11, wherein the component (A) and component (B) are combined so that a transparent solution is obtained when a varnish (having a solids content of 50% by weight) is prepared from the component (A) and component (B) in a 50/50 weight ratio using a mixed solvent comprising 85 parts by weight of toluene and 15 parts by weight of acetone.

14. The uncured film according to claim 11, which forms a heat-cured product having an elastic modulus of 0.4 to 2.7 GPa (25° C.), as measured by dynamic viscoelastic analysis (DMA) at a frequency of 10 Hz (tensile mode), and having a glass transition temperature of 150° C. or higher.

15. An interlayer dielectric film for printed wiring board obtained by using the uncured film according to claim 11.

16. An electronic part obtained by using the uncured film according to claim 11.
